(19) United States
(12) Patent Application Publication
(10) Pub. No.: US 2024/0248071 A1
(43) Pub. Date: Jul. 25, 2024

(54) SENSOR FOR MEASURING A GAS PROPERTY

(71) Applicant: Infineon Technologies AG, Neubiberg (DE)

(72) Inventors: Julia Isabel PEREZ BARRAZA, Unterhaching (DE); Marten OLDSEN, Tanjung Bungah (MY); Matthias EBERL, Taufkirchen (DE)

(21) Appl. No.: 18/414,733

(22) Filed: Jan. 17, 2024

(30) Foreign Application Priority Data
Jan. 24, 2023 (DE) 102023101637.7

(51) Int. Cl. G01N 33/00; H01L 21/74; H01L 21/768; H01L 23/00; H01L 23/498

(52) U.S. Cl. CPC G01N 33/005; H01L 21/743; H01L 21/76871; H01L 23/49838; H01L 23/49872; H01L 24/32; H01L 2224/32188

(57) ABSTRACT

Examples disclose a sensor for measuring a gas property, in particular a gas composition, more particularly a hydrogen level, wherein the sensor includes a semiconductor die, wherein the semiconductor die includes a measuring cavity, wherein a measuring sensor element is arranged in the measuring cavity, wherein the semiconductor die includes a contact pad, wherein the semiconductor die includes a buried conductor, wherein the buried conductor electrically connects the measuring sensor element to the contact pad, wherein a conductive bonding layer of the semiconductor die surrounds the measuring cavity for providing a conductive bonding surface, and wherein the buried conductor is insulated from the conductive bonding layer. Further examples, disclose methods for manufacturing a sensor.

### CROSS-REFERENCE TO RELATED APPLICATION

This application claims priority to German Patent Application No. 102023101637.7 filed on Jan. 24, 2023, the content of which is incorporated by reference herein in its entirety.

### TECHNICAL FIELD

The disclosure relates to a sensor for measuring a gas property and a method for manufacturing a sensor for measuring a gas property.

### BACKGROUND

There is an increasing demand for reducing the consumption of petroleum and shifting to using green energy. For example, hydrogen generated by wind turbines is considered as a possible green fuel for automotive applications.

Sensors may be required to detect any leaking hydrogen to avoid the formation of Oxyhydrogen.

A highly sensitive hydrogen sensor to be operated at room temperature is disclosed in DE 10 2004 033597 A1. However, cars may be operated at temperatures well below and above room temperature.

A further sensor for measuring a gas property is disclosed in DE 10 2020 134 366 A1. The sensor for measuring a gas property, in particular a gas composition, more particularly a hydrogen level, comprises the semiconductor die, wherein the semiconductor die comprises a reference cavity and a measuring cavity. A reference sensor element is arranged in the reference cavity and a measuring sensor element is arranged in the measuring cavity. The reference cavity is sealed from ambient gas and the measuring cavity is fluidly connected to ambient gas. A fluid connection may relate to a connection allowing the passing of liquids and/or gas. For example, the reference cavity may be covered with a membrane allowing diffusion of gas into the measuring cavity.

### SUMMARY

There may be a need for a sensor for measuring a gas property for automotive applications being simpler to manufacture and a corresponding method for manufacturing one or more sensors for measuring a gas property.

Subject-matter as defined in the independent claims is provided. Further implementations are described in the dependent claims.

Examples disclose a sensor for measuring a gas property, in particular a gas composition, more particularly a hydrogen level, wherein the sensor includes a semiconductor die, wherein the semiconductor die includes a measuring cavity, wherein a measuring sensor element is arranged in the measuring cavity, wherein the semiconductor die includes a contact pad, wherein the semiconductor die includes a buried conductor, wherein the buried conductor electrically connects the measuring sensor element to the contact pad, wherein a conductive bonding layer of the semiconductor die surrounds the measuring cavity for providing a conductive bonding surface, and wherein the buried conductor is insulated from the conductive bonding layer.

Moreover, examples disclose a method for manufacturing one or more sensors for measuring a gas property, in particular a gas composition, in particular a hydrogen level, wherein the method includes: providing a semiconductor wafer substrate having a front side and a back side; providing a first dielectric layer on a front side of the semiconductor substrate; providing a buried conductor on the first dielectric layer; providing a second dielectric layer on the first dielectric layer and the buried conductor, only partially covering the buried conductor; providing a semiconductor layer; providing a mask, in particular a hard mask, on the semiconductor layer, etching at measuring cavity and optionally a reference cavity in the back side of the semiconductor wafer substrate to form a membrane; by etching, forming a measuring sensor element and optionally a reference sensor element from parts of the membrane and forming a conductive bonding layer surrounding the measuring cavity from the semiconductor layer by etching.

### BRIEF DESCRIPTION OF THE DRAWINGS

Implementations of the proposed sensor and the proposed method for manufacturing a sensor will now be explained with reference to the drawing. In the drawing

FIG. 1 shows a semiconductor wafer;

FIG. 2 shows a semiconductor wafer obtained by processing the semiconductor wafer of FIG. 1;

FIG. 3 shows a semiconductor wafer obtained by processing the semiconductor wafer of FIG. 2;

FIG. 4 shows a semiconductor wafer obtained by processing the semiconductor wafer of FIG. 3;

FIG. 5 shows a semiconductor wafer obtained by processing the semiconductor wafer of FIG. 4;

FIG. 6 shows a semiconductor wafer obtained by processing the semiconductor wafer of FIG. 5;

FIG. 7 shows a semiconductor wafer obtained by processing the semiconductor wafer of FIG. 6;

FIG. 8 shows a semiconductor wafer obtained by processing the semiconductor wafer of FIG. 7;

FIG. 9 shows a semiconductor wafer obtained by processing the semiconductor wafer of FIG. 8;

FIG. 10 shows a semiconductor wafer obtained by processing the semiconductor wafer of FIG. 9;

FIG. 11 shows a semiconductor wafer obtained by processing the semiconductor wafer of FIG. 10;

FIG. 12 shows a semiconductor wafer obtained by processing the semiconductor wafer of FIG. 11;

FIG. 13 shows a semiconductor wafer obtained by processing the semiconductor wafer of FIG. 12;

FIG. 14 shows a semiconductor wafer obtained by processing the semiconductor wafer of FIG. 13;

FIG. 15 shows the semiconductor wafer of FIG. 14 in a top view;

FIG. 16 shows a sensor for measuring a gas property;

FIG. 17 shows a sensor for measuring a gas property; and

FIG. 18 illustrates a method for manufacturing a sensor.

### DETAILED DESCRIPTION

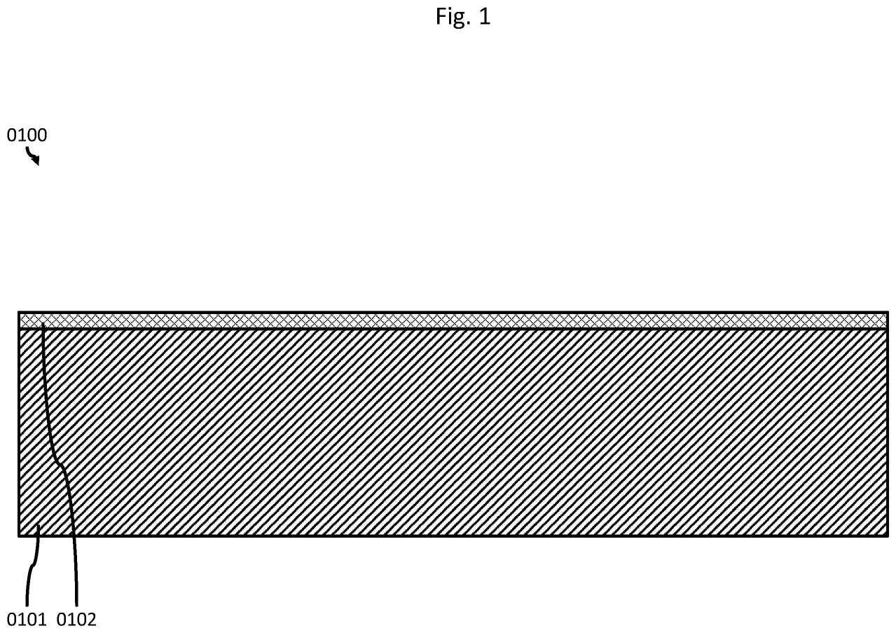

FIG. 1 shows a semiconductor wafer 0100 with a front side and a back side. A first dielectric layer 0102 may be provided on the front side of the semiconductor wafer 0101. Thus, the first dielectric layer 0102 is shown on top of a semiconductor substrate 0101. The semiconductor substrate 0101 may be formed from silicon. The first dielectric layer 0101 may be from silicon dioxide. In particular, the first dielectric layer 0101 may be formed from thermal silicon dioxide. Alignment markers may be formed in the first dielectric layer 0102. The alignment markers may be formed in the first dielectric layer 0102 using etching.

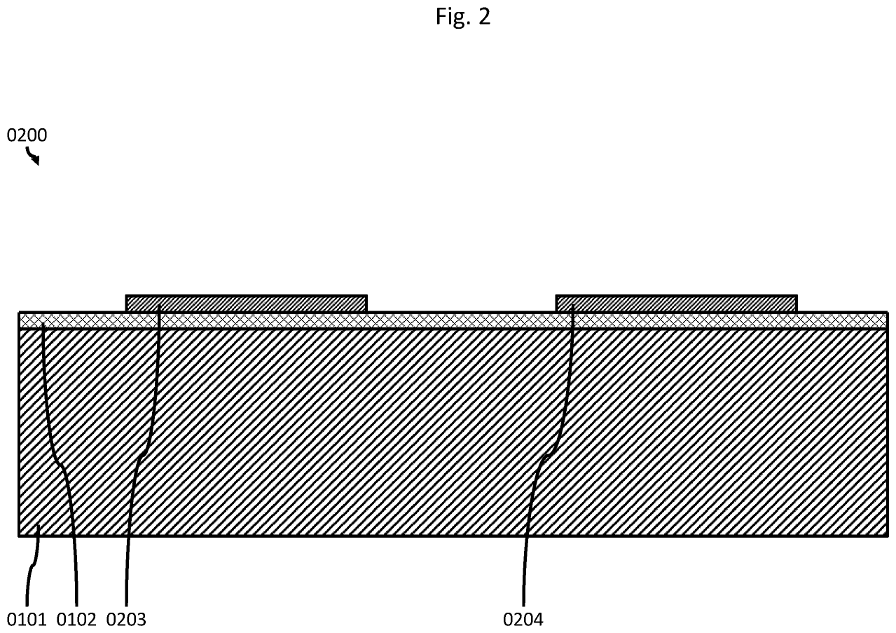

FIG. 2 shows the semiconductor wafer 0200 after processing the semiconductor wafer 0100. Processing may include providing (later) buried conductors 0203, 0204 on the first dielectric layer 0102. For example, a doped semiconductor layer may be deposited on the first dielectric layer 0102 and patterned afterwards to obtain the buried conductors 0203, 0204. In some examples, doped polysilicon may be deposited on the first dielectric layer 0102. In particular, doped polysilicon may be deposited using a CVD (chemical vapor deposition) process or it can be epitaxily grown.

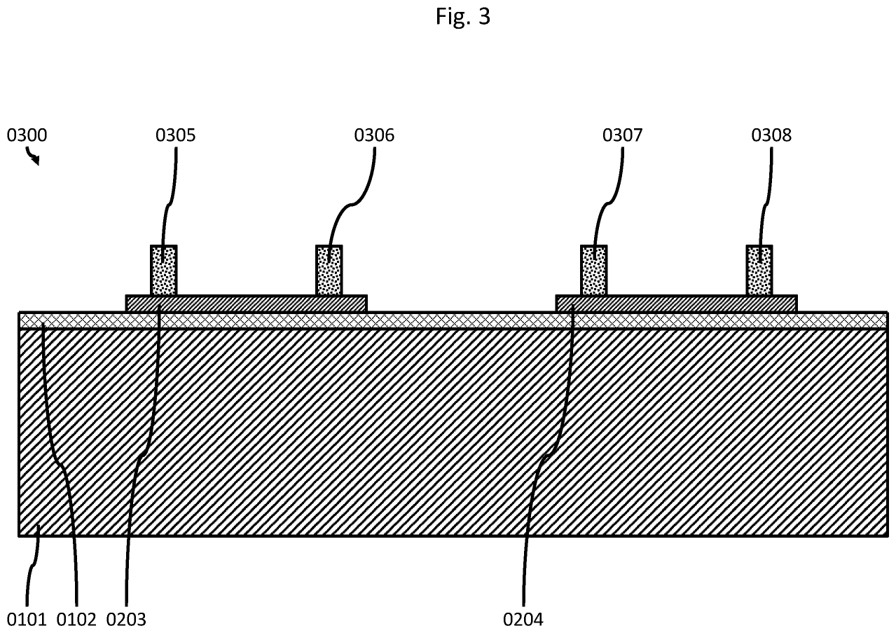

FIG. 3 shows the semiconductor wafer 0300 after further processing the semiconductor wafer 0200. Spacers 0305, 0306, 307, 308 may be provided on the buried conductors 0203, 0204.

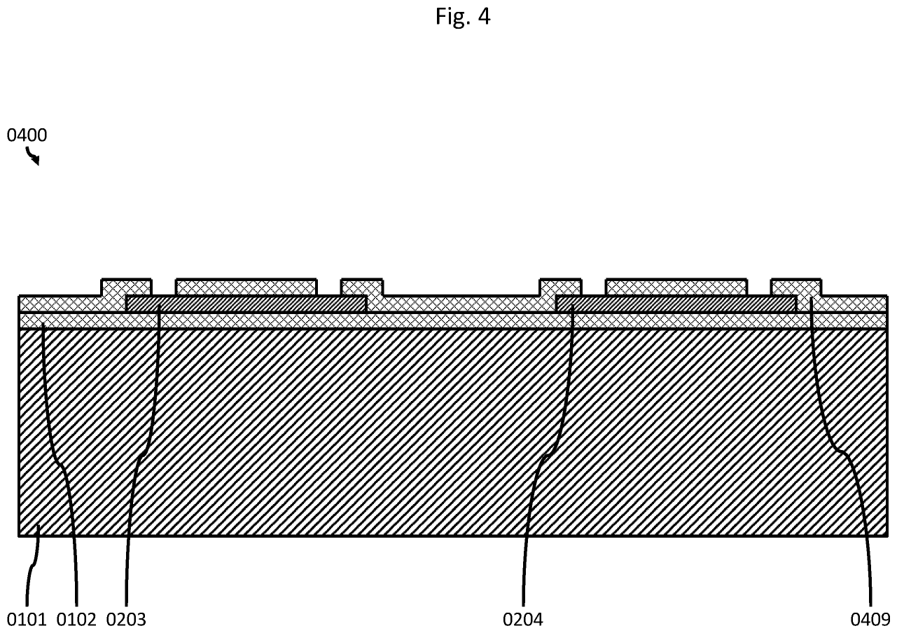

FIG. 4 shows the semiconductor wafer 0400 after depositing a second dielectric layer 0409 on the semiconductor wafer 0300 and removing the spacers 0305, 0306, 0307, 0308. The spacers 0305, 306, 307, 308 may ensure that the second dielectric layer 0409 only partially covers the buried conductors 0203, 204. Providing the second dielectric layer 0409 on the first dielectric layer 0102 and the buried conductors 0203, 0204, while only partially covering the buried conductors 0203, 0204 may be performed using a TEOS (tetraethoxysilane) process.

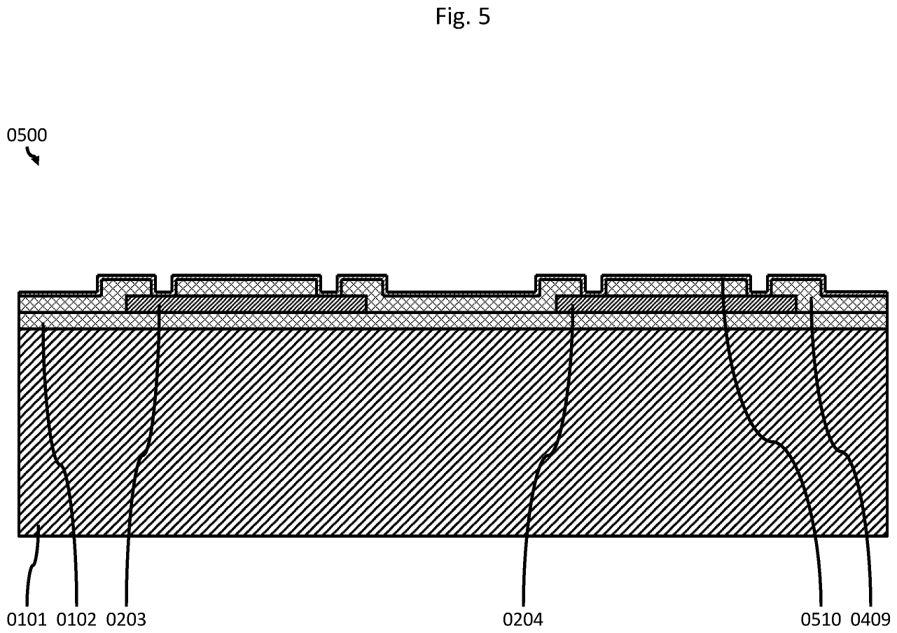

A seed layer 0510 may be provided on the semiconductor wafer 0400 to obtain the semiconductor wafer 0500 shown in FIG. 5. The seed layer 0510 may cover the second dielectric layer 0409 and the buried conductors 0203, 0204. The seed layer 0510 may be made from a semiconductor. In particular, the seed layer 0510 may be made from silicon. The seed layer 0510 may be very thin. In particular, the seed layer 0510 may have a thickness below 500 nm.

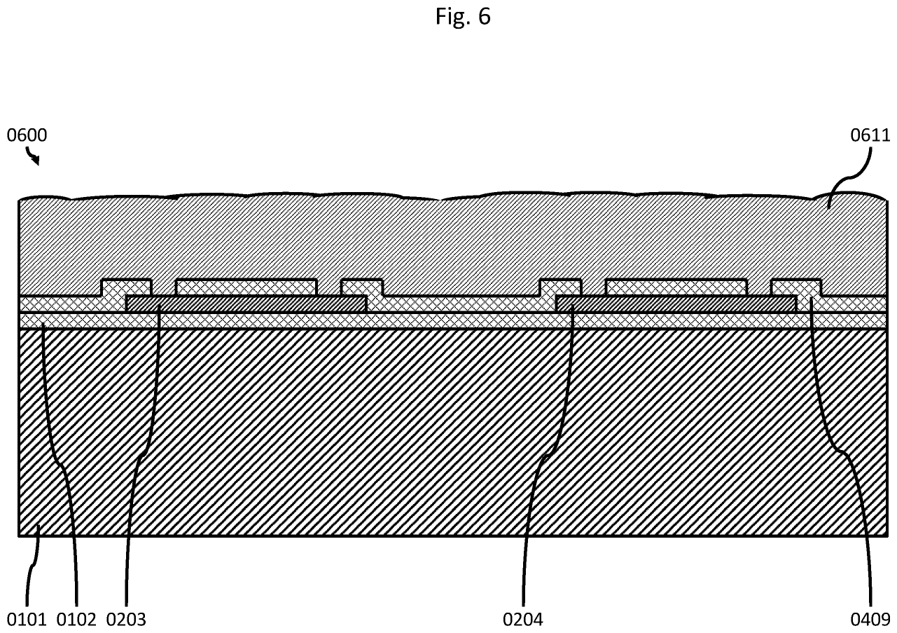

FIG. 6 shows the semiconductor wafer 0600 after processing the semiconductor wafer 0500. In particular, a semiconductor layer 0611 may be grown from the seed layer 0510. A process for epitactic deposition of the semiconductor may be used. The process may lead to a polycrystalline semiconductor layer 0611 having a larger grain size than the buried conductors 0203.

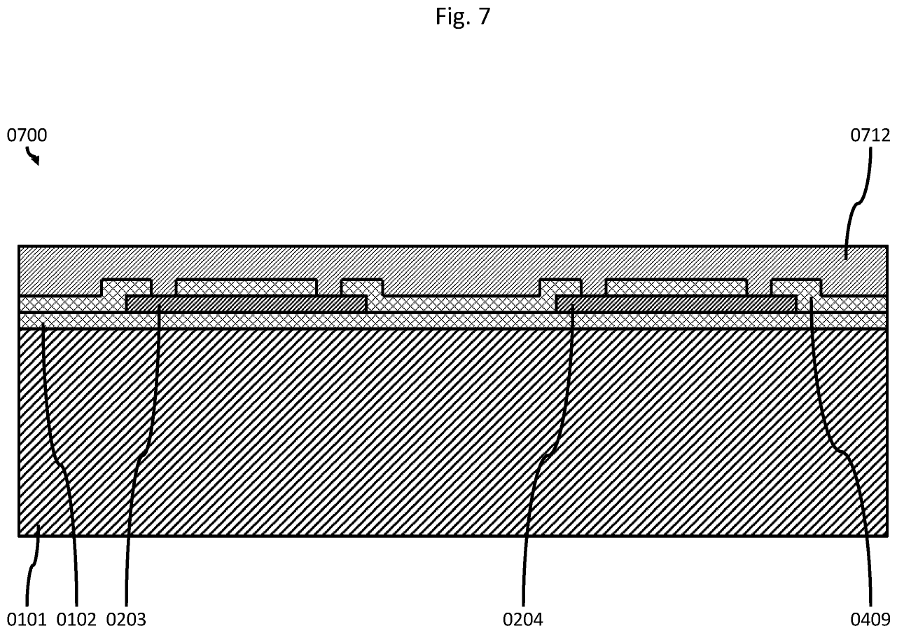

FIG. 7 shows the semiconductor wafer 700 after polishing the semiconductor layer 0611 to obtain a semiconductor layer 0711. A chemical mechanical polishing process may be used to obtain the semiconductor layer 0611. In some scenarios, an alignment marker may be provided after chemical mechanical polishing.

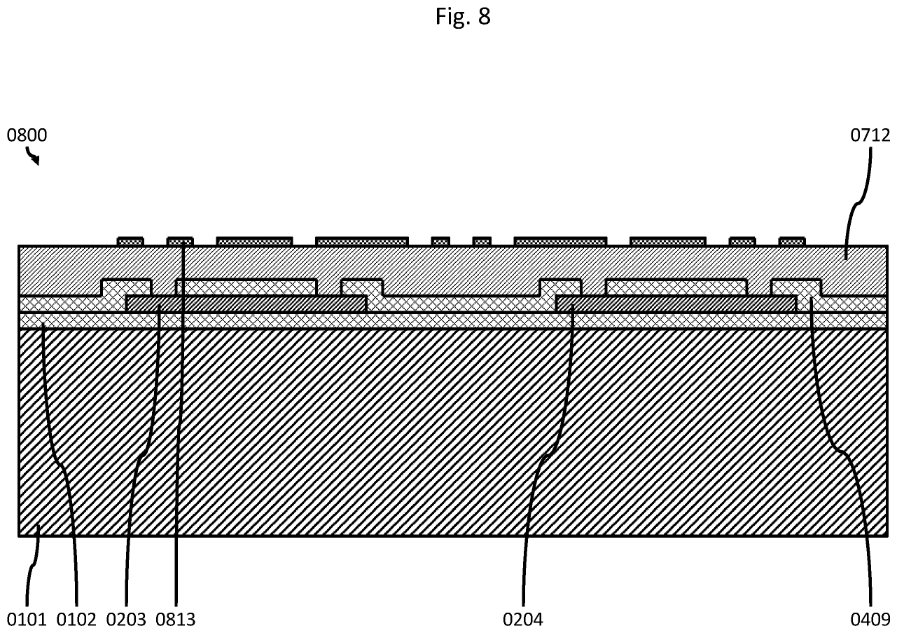

A mask 0813 may be provided on the semiconductor layer 0711 as shown in FIG. 8. The mask 0813 on the semiconductor wafer 0800 may allow for structuring the semiconductor layer 0711. The mask 0813 may be a hard mask.

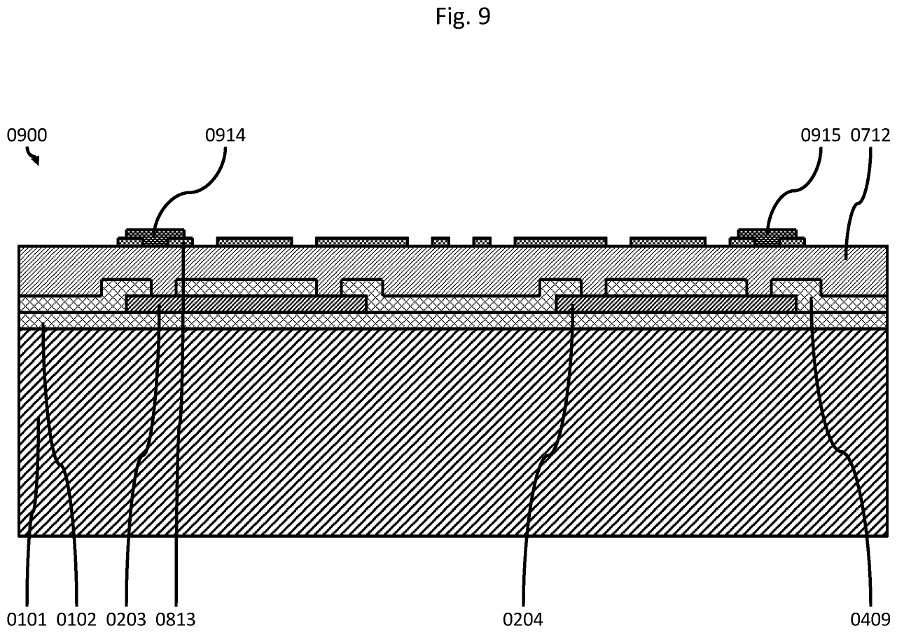

As shown in FIG. 9, contact pads 0914, 0915 may be provided. The contact pads 0914, 0915 may be provided by depositing a metal and patterning.

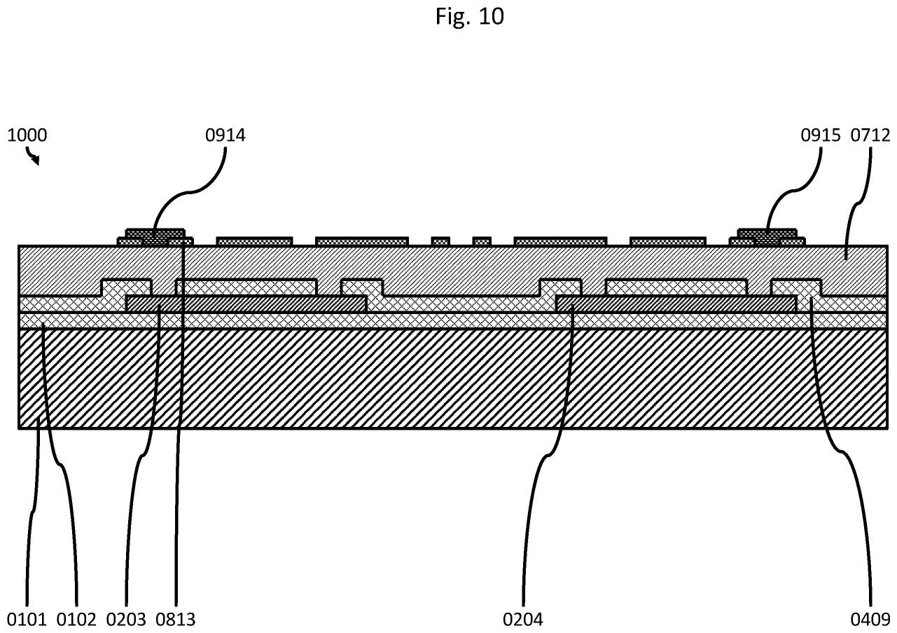

FIG. 10 shows the semiconductor wafer 1000 after the semiconductor substrate 0101 has been thinned. Backside grinding may be performed for thinning the semiconductor substrate 0101. After thinning, the semiconductor substrate 0101 may have a thickness below 590 μm, in particular below 490 μm, more particularly between 350 μm and 450 μm.

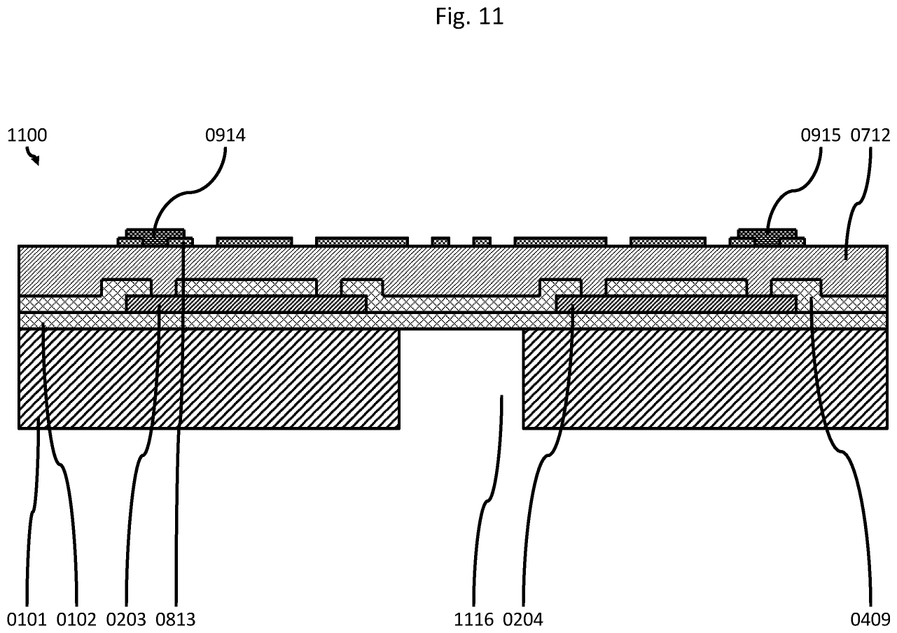

Further, the backside of the semiconductor substrate 0101 may be etched to form the cavity 1116 of the semiconductor wafer 1100 as shown in FIG. 11. In particular, the interface between the semiconductor substrate 0101 and the first dielectric layer 0102 may be used as an etch stop when etching the back side of the (thinned) semiconductor substrate. Thinning, in particular backside grinding, as explained with reference to FIG. 10 may reduce the time required for etching the cavity 1116 up to the interface between the semiconductor substrate 0101 and the first dielectric layer 0102.

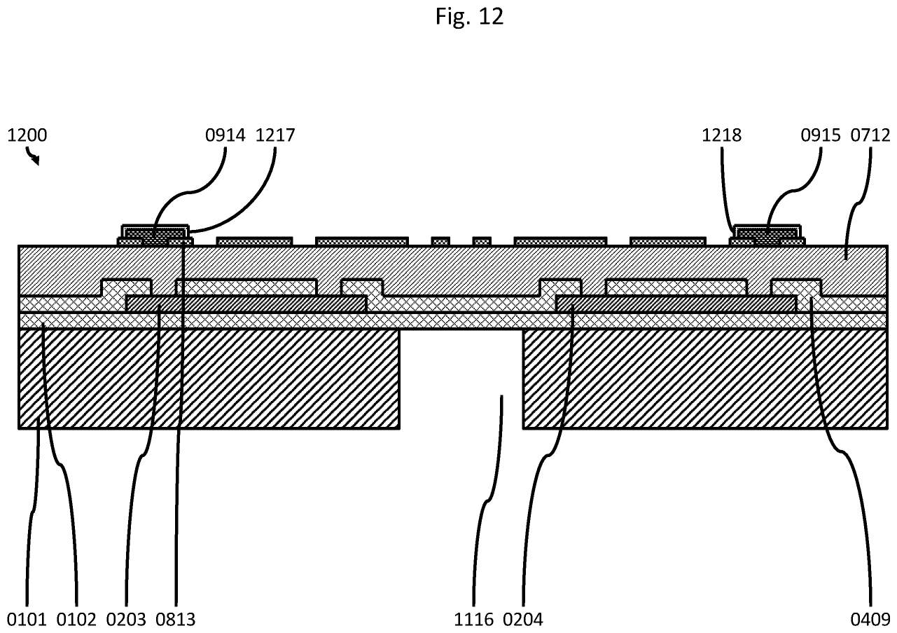

A protection layer 1217, 1218 may be provided on the contact pads 0914, 0915 to obtain the semiconductor wafer 1200 depicted in FIG. 12.

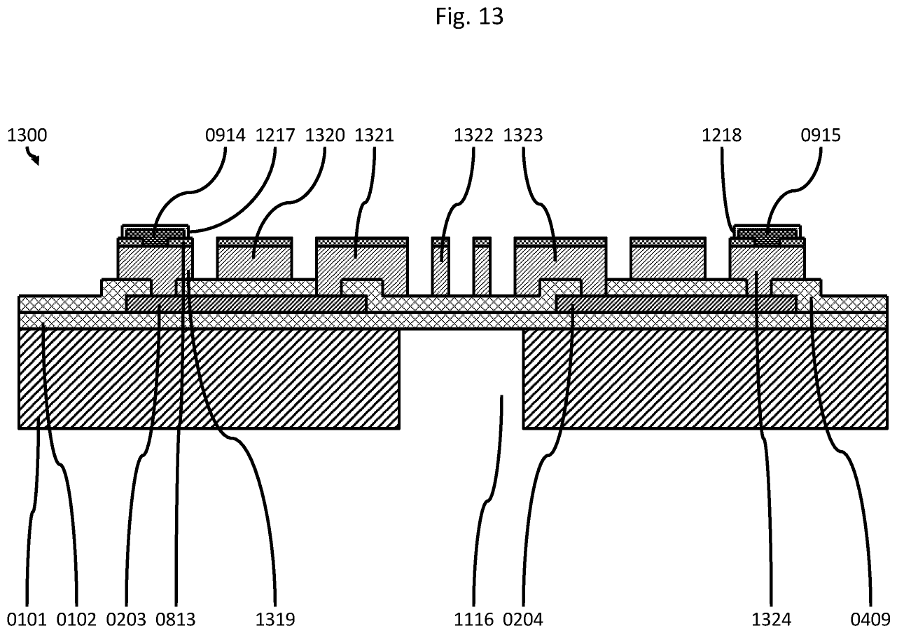

FIG. 13 shows the semiconductor wafer 1300 after etching through the semiconductor layer 0712 to insulate portions 1320, 1321, 1322, 1323 of the semiconductor layer 0712 from each other. In particular, a measuring sensor element 1322 is formed above the cavity 1116 and a conductive bonding layer 1320 is insulated from the buried conductors 0203, 0204. The protection layer 1217, 1218 may protect the contact pads 0914, 0915 while etching through the semiconductor layer 0712.

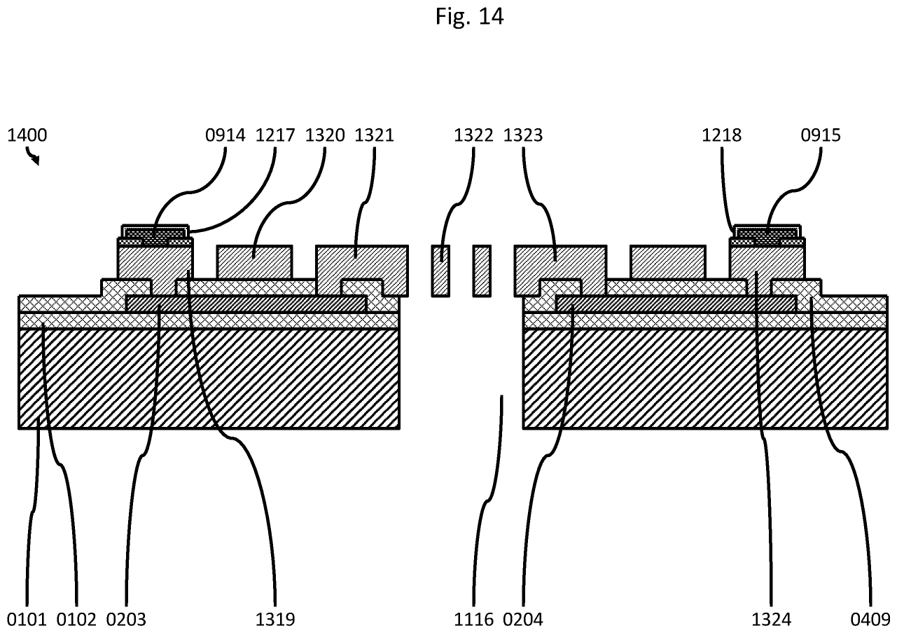

As shown in FIG. 14, the mask 0813 and the portion of the first dielectric layer 0102 and the second dielectric layer 0409 in the cavity 1116 may be removed to obtain the semiconductor wafer 1400. In particular, the measurement sensor element 1322 may be free from oxide. This may render the measurement sensor element 1322 less sensible to humidity. The active thickness of the measurement sensor element 1322 may be above 2 μm, in particular above 2.5 μm, more particularly around 3 μm. The thickness may be changed by changing the thickness of the semiconductor layer 0712. Changing the thickness of the semiconductor layer 0712 may have little influence on the time required for manufacturing the sensor. In particular, thicker semiconductor layers 0712 may require less time than providing deeper implantation regions as used in legacy processes for manufacturing sensors.

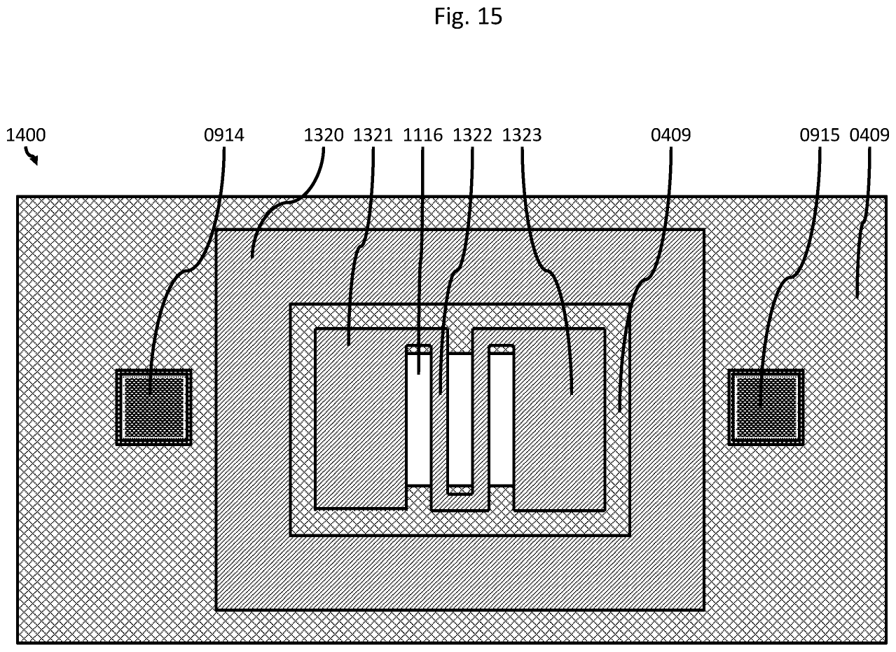

FIG. 15 shows a top view of the semiconductor wafer 1400. A conductive bonding layer 1320 surround the cavity 1116 for providing a conductive bonding surface.

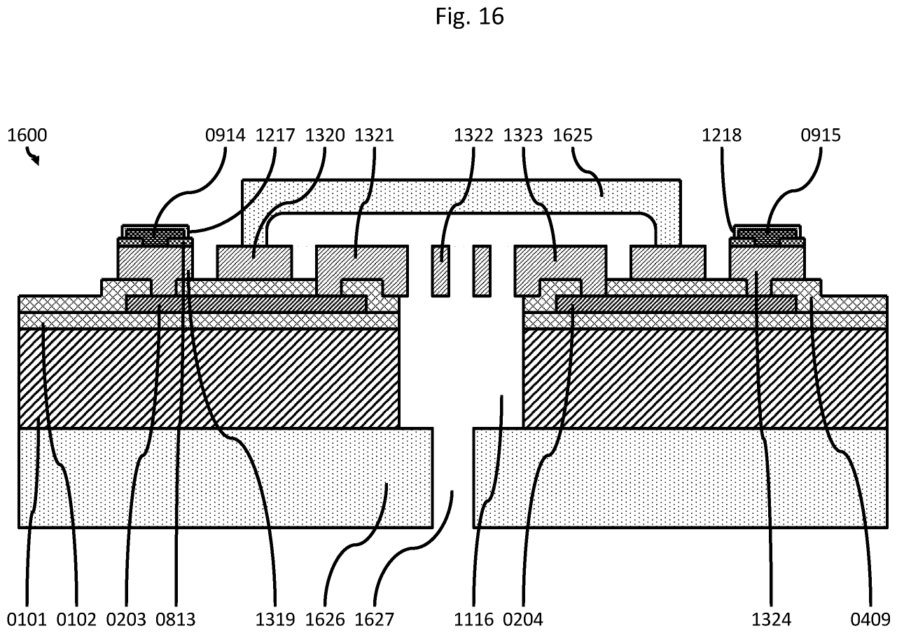

FIG. 16 shows a side view of the sensor 1600 wherein a covering 1625 is bonded to the bonding layer by anodic bonding. Likewise a covering 1626 is provided on the backside of the semiconductor substrate 0101. The covering 1626 is provided with a conduit 1627 to allow gas to be measured to enter the cavity 1116. In some scenarios, the conduit may be alternatively or in addition be provided in the covering 1625. Before or after providing the coverings 1625, 1626, dicing may take place. Thus, the reference numeral 1600 may refer to a semiconductor wafer comprising a plurality of sensors for measuring a gas property or a single sensor for measuring a gas property.

The propose method for manufacturing a sensor for measuring a gas property may require less process steps than known processes. In particular, it offers an increased flexibility with respect to the thickness of the measuring sensor element.

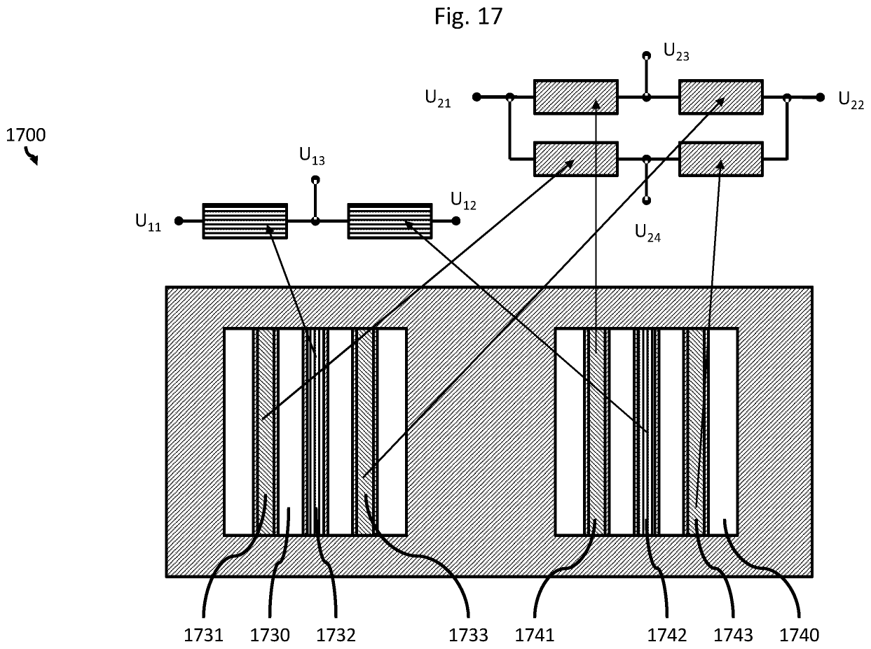

FIG. 17 shows a sensor 1700 for measuring a gas property which may have been manufactured using a method as explained with reference to FIGS. 1 to 16. The sensor 1700 may be in particular configured for measuring a gas composition, for example, a hydrogen level. A reference cavity 1730 and a measuring cavity 1740 are provided. The reference cavity 1730 and the measuring cavity 1740 may have been manufactured in the same way as the cavity 1116 as explained hereinbefore. Three reference sensor elements 1731, 1732, 1733 may be provided in the reference cavity 1730 and corresponding three measuring sensor elements 1741, 1742, 1743 may be provided in a reference cavity. The sensor elements 1731, 1732, 1733, 1741, 1742, 1743 may have been manufactured similar to the sensor element 1322.

The reference cavity 1740 is sealed from ambient gas. In particular, the reference cavity 1740 may be hermetically sealed from ambient gas. On the other hand, the measuring cavity 1730 is fluidly connected to ambient gas. In particular, a conduit may be provided for the purpose.

The reference sensor elements 1731, 1732, 1733 and the measuring sensor elements 1741, 1742, 1743 may be formed in the same semiconductor layer 0702. This may facilitate manufacturing of the sensor. Moreover, it may lead to reference sensor elements having the same property as the measuring sensor elements.

Sensors for measuring a gas property, which may also be called gas sensors, may have a cross-sensitivity to different environment characteristics, such as humidity, temperature, flow and concentration of the gas to be sensed. Typically, dedicated sensors for these additional properties may have to be included in order to differentiate the signal of interest. For example, the complementary temperature sensor may have to be added. This may lead to a complex device, where different dice or sensing elements have to be combined inside the package.

The sensors as disclosed herein may be fabricated with two identical sensing elements (e.g., the reference sensor element and the measuring sensor element) in one die. One element (e.g., the measuring sensor element) is exposed to the ambient of interest and the other element (e.g., the reference sensor element) is enclosed within a hermetically sealed cavity (e.g., the reference cavity). Hence, the package complexity may be reduced. Further, the device sensitivity may be improved.

For example, a differential read out between the two sensor elements (e.g., the reference sensor element and the measuring sensor element) may significantly reduce or even eliminate cross-sensitivity to temperature, as well as other sources of error and operational drift.

As also shown in FIG. 17, a reference sensor element and a measuring sensor element may be electrically connected to form a half bridge. In particular, the reference sensor element 1742 and the measuring sensor element 1732 may be electrically connected to form half bridge. This may facilitate reading out the sensor.

The sensor 1700 may comprise at least two reference sensor elements and at least two measuring sensor elements forming a full bridge. In particular, one reference sensor element 1741 of the two reference sensor elements 1741, 1743 may be electrically connected with a first node U23 to a first node U23 of one measuring sensor element 1733 of the two measuring sensor elements 1731, 1733 and with a second node U21 to a second node U21 of the other measuring sensor element 1731 of the two measuring sensor elements 1731 and 1733. The other reference sensor element 1743 of the two reference sensor elements 1741, 1743 may be electrically connected with the first node U24 to a first node U24 of the other measuring sensor element 1731 of the two measuring sensor elements 1731 and 1733 and with a second node U22 to the second node U22 of the measuring sensor element 1733 of the two measuring sensor elements 1731 and 1733.

Due to the provision of the reference sensor elements in the reference cavity, the sensor 1700 may be suitable to be operated between −40° C. and 150° C.

In examples, the reference sensor element and the measuring sensor element may be formed as corresponding membranes. Alternatively, as shown in the figures, the reference sensor elements and the measuring sensor elements may be formed as corresponding wires. These may be linear wires or meander wires.

The reference sensor element 1742 and the measuring sensor element 1732 may comprise a catalytic layer for reacting with gas molecules. In particular, the catalytic layer may be formed from platinum and/or palladium for reacting with hydrogen molecules. The catalytic layer may also be formed using additional are different noble methods.

As shown in FIG. 17, the sensor 1700 comprises at least two reference sensor elements 1741, 1742 and at least two measuring sensor elements 1731, 1732. The reference sensor element 1741 corresponds to the measuring sensor element 1731. Likewise, the reference sensor element 1742 corresponds to the measuring sensor element 1732. The reference sensor element 1741 and the measuring sensor element 1731 may be configured for using different measurement principle than the reference under element 1742 and the second measuring sensor element 1732.

For example, the reference sensor element 1741 and the measuring sensor element 1731 may be configured for measuring a gas concentration via thermal conductivity. For example, the reference sensor element 1741 and the measuring sensor element 1731 may comprise silicon wires etched from a thin membrane. The silicon wires may be doped to increase the electrical conductance.

On the other hand, the reference sensor element 1742 and the measuring sensor element 1732 may be configured to measure a gas concentration via catalytic combustion. For example, the catalytic layer may react with gas molecules which may induce a modification of the electrical properties of the reference sensor element 1742 and the measuring sensor element 1732. By applying a current and heating the respective reference sensor element 1742 and measuring sensor element 1732, the gas molecules may be released again and the sensor may be reset.

According to FIG. 17, a half-bridge electric circuit may be used for the sensor elements which use a catalytic layer technique for detecting a gas concentration and a full-bridge electric circuit may be used for the sensor elements which us a thermal conductivity technique for detecting the gas concentration. This may lead to similar signal levels for both types of sensor elements and facilitate the combination of the measurement results obtained by both techniques.

The combination of two measuring principles within one sensor may further enhance the precision of the sensor. Further gas sensing principles and/or mixture of those sensing principles may be used as well.

The proposed sensor may be particularly useful for automotive powertrains based on hydrogen fuel cells. For example, a sensor of the described type may be located near an exhaust of the fuel cell, in order to control the fuel cell. Heretofore, the sensor may be configured to determine an H2 content of 0 to 40 percent.

Furthermore, the sensor may be located next to the high pressure H2 tank. The sensor may be configured for sensing an H2 leakage. For this purpose, the sensor may have a sensitivity for a concentration of 0 to 4% H2. In other examples, the sensor may be located close to a battery pack. The sensor may be configured for detecting out gassing of H2 due to the battery pack being overloaded and/or damaged. For this purpose, the sensor may detect H2 with a concentration of 0 to 4%.

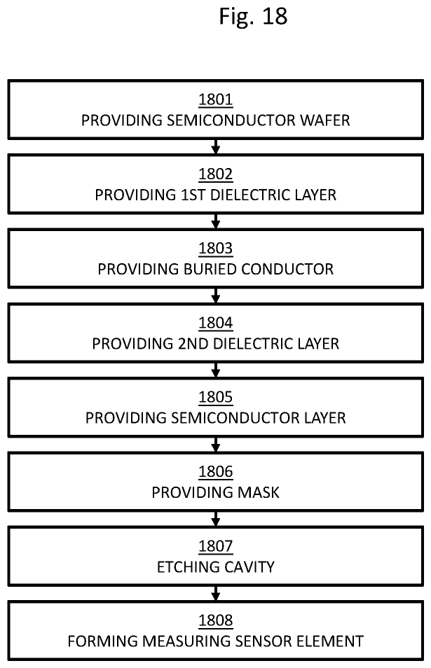

FIG. 18 illustrates a method for manufacturing one or more sensors for measuring a gas property, in particular a gas composition, in particular a hydrogen level. The method comprises providing a semiconductor substrate having a front side and a back side at 1801. At 1802, a first dielectric layer is provided on a front side of the semiconductor substrate. Afterwards, the method prescribes providing a buried conductor on the first dielectric layer as indicated with box 1803. At 1804, a second dielectric layer is provided on the first dielectric layer and the buried conductor, wherein the second dielectric layer only partially covers the buried conductor. A semiconductor layer is provided on the second dielectric layer is provided at 1805. At 1806, a mask, in particular a hard mask, is provided on the semiconductor layer. A measuring cavity and optionally a reference cavity is etched in the back side of the semiconductor substrate at 1807 to form a membrane. At 1808, the method prescribes, by etching, forming a measuring sensor element and optionally a reference sensor element from parts of the membrane and forming a conductive bonding layer surrounding the measuring cavity from the semiconductor layer by etching.

### Aspects

In particular, the following ASPECTS are disclosed:

ASPECT 1. A sensor (1600) for measuring a gas property, in particular a gas composition, more particularly a hydrogen level, wherein the sensor (1600) comprises a semiconductor die, wherein the semiconductor die comprises a measuring cavity (1116), wherein a measuring sensor element (1322) is arranged in the measuring cavity (1116), wherein the semiconductor die (0101) comprises a contact pad (0914, 0915), wherein the semiconductor die comprises a buried conductor (0203, 0204), wherein the buried conductor (0203, 0204) electrically connects the measuring sensor element (1322) to the contact pad (0914, 0915), wherein a conductive bonding layer (1320) of the semiconductor die surrounds the measuring cavity (1116) for providing a conductive bonding surface, and wherein the buried conductor (0203, 0204) is insulated from the conductive bonding layer (1320).

ASPECT 2. The sensor (1700) of ASPECT 1, wherein the semiconductor die comprises a reference cavity (1740), wherein a reference sensor element (1741, 1742, 1743) is arranged in the reference cavity (1740), wherein the reference cavity (1740) is sealed from ambient gas, wherein the measuring cavity (1730) is fluidly connected to ambient gas.

ASPECT 3. The sensor (1600) of ASPECTS 1 or 2, wherein the measuring sensor element (1322) and/or the reference sensor element is made from a first type of polysilicon.

ASPECT 4. The sensor (1600) of ASPECT 3, wherein the conductive bonding layer (1320) is made from the first type of polysilicon.

ASPECT 5. The sensor (1600) of any one of Aspects 1 to 4, wherein the buried conductor (0203, 0204) is made from a second type of polysilicon.

ASPECT 6. The sensor (1600) of ASPECT 5, wherein the second type of polysilicon has a larger grain size than the first type of polysilicon.

ASPECT 7. The sensor (1600) of any one of Aspects 1 to 6, wherein the buried conductor (0203, 0204) is insulated from the conductive bonding layer by a dielectric material.

ASPECT 8. The sensor (1600) of Aspect 7, wherein the dielectric material comprises silicon oxide.

ASPECT 9. The sensor (1600) of any one of Aspects 1 to 8, wherein the sensor (1600) comprises a covering (1625, 1626) for covering the measuring cavity (1116) and optionally sealing the reference cavity.

ASPECT 10. The sensor (1600) of any one of Aspects 1 to 9, wherein the covering (1625) is bonded to the conductive bonding layer (1320) by anodic bonding.

ASPECT 11. The sensor (1600) of any one of Aspects 1 to 10, wherein the measuring sensor element (1322) and/or the reference sensor element is formed as one piece with the semiconductor die.

ASPECT 12. The sensor (1600) of any one of Aspects 1 to 11, wherein the reference cavity is filled with a gas, in particular a reference gas.

ASPECT 13. The sensor (1600) of any one of Aspects 1 to 12, wherein the reference sensor element and the measuring sensor element (1322) have the same structure.

ASPECT 14. The sensor (1600) of any one of Aspects 1 to 13, wherein the semiconductor die comprises an integrated circuit and wherein the measuring sensor element (1322) and/or the reference sensor element are part of the integrated circuit.

ASPECT 15. The sensor (1700) of any one of Aspects 2 to 14, wherein the measuring sensor element (1732) and the reference sensor element (1742) are electrically connected to form a half bridge.

ASPECT 16. The sensor (1700) of any one of Aspects 2 to 15, wherein the sensor (1700) comprises at least two reference sensor elements (1741, 1743) and at least two measuring sensor elements (1731, 1733), wherein one (1741) of the two reference sensor elements (1741, 743) is electrically connected with a first node to a first node of one (1731) of the two measuring sensor elements (1731, 1733) and with a second node to a second node of the other one (1733) of the two measuring sensor elements (1731, 1733, wherein the other one (1743) of the two reference sensor elements (1741, 1743) is electrically connected with a first node to a first node of the other one (1733) of the two measuring sensor elements (1731, 1733) and with a second node to the second node of the one (1731) of the two measuring sensor elements (1731, 1733).

ASPECT 17. The sensor of any one of Aspects 2 to 16, wherein the reference sensor element and the measuring sensor element are formed as corresponding membranes.

ASPECT 18. The sensor (1700) of any one of Aspects 2 to 17, wherein the reference sensor element (1741, 1742, 1743) and the measuring sensor element (1731, 1732, 1733) are formed as corresponding wires.

ASPECT 19. The sensor (1700) of any one of Aspects 1 to 18, wherein the measuring sensor element (1732) and/or the reference sensor element (1742) comprise a catalytic layer for reacting with gas molecules.

ASPECT 20. The sensor (1700) of any one of Aspects 1 to 19, wherein the measuring sensor element (1731, 1732, 1733) and/or the reference sensor element (1741, 1742, 1743) is free of silicon oxide.

ASPECT 21. The sensor (1700) of any one of Aspects 2 to 20, wherein the sensor comprises at least two reference sensor elements and at least two measuring sensor elements; wherein a first reference sensor element of the at least two reference sensor elements corresponds to a first measuring sensor element of the at least two measuring sensor elements and a second reference sensor element of the at least two reference sensor elements corresponds to a second measuring sensor element of the at least two measuring sensor elements, and wherein the first reference sensor element and the first measuring sensor element are configured for using a different measurement principle than the second reference sensor element and the second measuring sensor element.

ASPECT 22. A method for manufacturing one or more sensors for measuring a gas property, in particular a gas composition, in particular a hydrogen level, wherein the method comprises: providing (1801) a semiconductor substrate (0101) having a front side and a back side; providing (1802) a first dielectric layer (0102) on a front side of the semiconductor substrate (0101); providing (1803) a buried conductor on the first dielectric layer; providing (1804) a second dielectric layer on the first dielectric layer and the buried conductor, only partially covering the buried conductor, providing (1805) a semiconductor layer, providing (1806) a mask, in particular a hard mask, on the semiconductor layer, etching (1807) at measuring cavity and optionally a reference cavity in the back side of the semiconductor substrate (0101) to form a membrane; by etching, forming (1808) a measuring sensor element and optionally a reference sensor element from parts of the membrane and forming a conductive bonding layer surrounding the measuring cavity from the semiconductor layer by etching.

ASPECT 23. The method of ASPECT 22, further comprising removing the mask for providing a conductive bonding surface.

ASPECT 24. The method of ASPECTS 22 or 23, further comprising bonding, in particular anodic bonding, at least one covering wafer to the semiconductor wafer for covering the measuring cavity and optionally sealing the reference cavity.

ASPECT 25. The method of any one of Aspects 22 to 24, further comprising: depositing a catalytic material on the conductive regions to form a catalytic layer for reacting with gas molecules.

ASPECT 26. The method of any one of Aspects 22 to 25, wherein providing a semiconductor layer comprises: providing a seed layer on the second dielectric layer, growing the semiconductor layer from the seed layer.

ASPECT 27. The method of ASPECT 26, further comprising performing chemical mechanical polishing after growing the semiconductor layer.

While this implementation has been described with reference to illustrative implementations, this description is not intended to be construed in a limiting sense. Various modifications and combinations of the illustrative implementations, as well as other implementations of the implementation, will be apparent to persons skilled in the art upon reference to the description. It is therefore intended that the appended claims encompass any such modifications or implementations.

1. A sensor for measuring a gas property, the sensor comprising:
a semiconductor die,
wherein the semiconductor die comprises:
a measuring cavity,
wherein a measuring sensor element is arranged in the measuring cavity,
a contact pad,
a buried conductor,
wherein the buried conductor electrically connects the measuring sensor element to the contact pad, and
wherein a conductive bonding layer surrounding the measuring cavity for providing a conductive bonding surface,
wherein the buried conductor is insulated from the conductive bonding layer.

2. The sensor of claim 1, wherein the semiconductor die comprises a reference cavity,
wherein a reference sensor element is arranged in the reference cavity,
wherein the reference cavity is sealed from ambient gas, and
wherein the measuring cavity is fluidly connected to ambient gas.

3. The sensor of claim 2, wherein at least one of the measuring sensor element or the reference sensor element is made from a first type of polysilicon.

4. The sensor of claim 3, wherein the conductive bonding layer is made from the first type of polysilicon.

5. The sensor of claim 4, wherein the buried conductor is made from a second type of polysilicon.

6. The sensor of claim 5, wherein the second type of polysilicon has a larger grain size than the first type of polysilicon.

7. The sensor of claim 1, wherein the buried conductor is insulated from the conductive bonding layer by a dielectric material.

8. The sensor of claim 7, wherein the dielectric material comprises silicon oxide.

9. The sensor of claim 2, wherein the sensor comprises a covering configured to one or more of:
cover the measuring cavity, or
seal the reference cavity.

10. The sensor of claim 9, wherein the covering is bonded to the conductive bonding layer by anodic bonding.

11. The sensor of claim 2, wherein one or more of the measuring sensor element or the reference sensor element is formed as one piece with the semiconductor die.

12. The sensor of claim 2, wherein the semiconductor die comprises an integrated circuit, and
wherein one or more of the measuring sensor element or the reference sensor element are part of the integrated circuit.

13. The sensor of claim 2, wherein the measuring sensor element and the reference sensor element are electrically connected to form a half bridge.

14. The sensor of claim 2, wherein the sensor comprises at least two reference sensor elements and at least two measuring sensor elements,
wherein one of the at least two reference sensor elements is electrically connected with a first node to a first node of one of the at least two measuring sensor elements and with a second node to a second node of another one of the at least two measuring sensor elements,
wherein the other one of the at least two reference sensor elements is electrically connected with a first node to a first node of the other one of the at least two measuring sensor elements and with a second node to the second node of the one of the at least two measuring sensor elements.

15. The sensor of claim 2, wherein at least one of the measuring sensor element or the reference sensor element comprises a catalytic layer for reacting with gas molecules.

16. The sensor of claim 2, wherein at least one of the measuring sensor element or the reference sensor element is free of silicon oxide.

17. The sensor of claim 2, wherein the sensor comprises at least two reference sensor elements and at least two measuring sensor elements,
wherein a first reference sensor element of the at least two reference sensor elements corresponds to a first measuring sensor element of the at least two measuring sensor elements and a second reference sensor element of the at least two reference sensor elements corresponds to a second measuring sensor element of the at least two measuring sensor elements, and
wherein the first reference sensor element and the first measuring sensor element are configured for using a different measurement principle than the second reference sensor element and the second measuring sensor element.

18. A method for manufacturing one or more sensors for measuring a gas property, wherein the method comprises:
providing a semiconductor substrate having a front side and a back side;
providing a first dielectric layer on the front side of the semiconductor substrate;
providing a buried conductor on the first dielectric layer;
providing a second dielectric layer on the first dielectric layer and the buried conductor, wherein the second dielectric layer only partially covers the buried conductor;
providing a semiconductor layer;
providing a mask, on the semiconductor layer;
etching at least one of a measuring cavity or a reference cavity in the back side of the semiconductor substrate to form a membrane;
by etching, forming at least one of a measuring sensor element or a reference sensor element from parts of the membrane; and
forming a conductive bonding layer surrounding the measuring cavity from the semiconductor layer by etching.

19. The method of claim 18, further comprising:
removing the mask for providing a conductive bonding surface.

20. The method of claim 18, wherein providing a semiconductor layer comprises:
providing a seed layer on the second dielectric layer, and
growing the semiconductor layer from the seed layer.
